(19) United States
(12) Patent Application Publication
(10) Pub. No.: US 2025/0050134 A1
(43) Pub. Date: Feb. 13, 2025

(54) MARKING ARRANGEMENT, MEDICAL DEVICE, AND SYSTEM FOR RECONSTRUCTING A PLANNED TRAJECTORY

(71) Applicant: NUCLETRON OPERATIONS B.V., Veenendaal (NL)

(72) Inventor: Farahnaz LIE-KWIE-SJOE, Veenendaal (NL)

(21) Appl. No.: 18/720,070

(22) PCT Filed: Dec. 16, 2022
(86) PCT No.: PCT/NL2022/050727

§ 371 Date: Jun. 14, 2024

(30) Foreign Application Priority Data
Dec. 16, 2021 (NL) 2030157

(51) Int. Cl. A61N 5/10; A61B 34/10; A61B 90/00

(52) U.S. Cl. CPC A61N 5/1039; A61N 5/1007; A61B 2034/107; A61B 2090/3954

(57) ABSTRACT

The invention relates to a marking arrangement (10) for marking positions of a medical device along a source trajectory (200), comprising a plurality of markers arranged in series along the source trajectory. Each marker (100) is aligned with the source trajectory and made of a first material and comprises a coating (110), wherein the coating is made of a second material disposed on a surface of the marker. A thickness (T) of the coating is smaller than 1 micrometer, and an interdistance (S) between each marker is smaller than 3 millimeter. The invention further relates to a system for reconstructing a planned trajectory of a medical device, comprising a detection module arranged for detecting the marking arrangement (10) and a treatment calculation module arranged for determining trajectories and dwell times.

### FIELD OF THE INVENTION

The invention relates to a marking arrangement for marking positions of a medical device along a source trajectory, a device comprising the marking arrangement, and a system for reconstructing a planned trajectory of a medical device.

### BACKGROUND

During high-dose-rate (HDR) brachytherapy treatment, one or more radioactive sources are introduced into or adjacent to a target volume of a patient, for example, a tumor. The radioactive sources may be introduced by using an afterloader device. Generally, the afterloader device is used to deliver the radioactive sources to a region inside of the patient for a given period of time at pre-determined dwell positions. Each radioactive source may be introduced via a cable, e.g. connected to a catheter, or for positioning within a brachytherapy applicator that is pre-positioned inside of the patient.

Besides accurate positioning of the applicator near the target area in the body, it is important that radioactive sources are introduced into the body along a carefully planned trajectory within the applicator. For example, if a target area is near a healthy organ, an important artery or nerve, or bony structures, the trajectory to the target area may need to avoid these tissue structures, e.g. by a margin of safety. This may also occur when multiple radioactive sources are to be positioned into or near a target area, which may require a safe distance between corresponding multiple planned trajectories, e.g. to prevent overtreatment.

Accordingly, one or more optimal trajectories can be defined computationally before the intervention, which is based on the type of treatment to be administrated and on information derived e.g. from computed tomography (CT) imaging and/or magnetic resonance imaging (MRI) of the patient's body, depending on the type of metastasis in the target area, and the anatomical structures expected around the target area that need to be avoided by the trajectory. For example, bony structures, the lungs, blood vessels and the applicator may be better visible with CT imaging, while MRI may be more suitable for muscle structures, the heart, and nerves.

During the intervention, it is an object to reconstruct the optimal planned trajectories, e.g. as stored in an Applicator Library File, as closely as possible. To determine the position of a radioactive source, when moving the radioactive source to the target area, it can be equipped with markers that can be visualized with CT imaging and/or MRI. By using a computer or other processing unit, a 3-dimensional position of a visualized marker can be mapped with a corresponding entry in the Applicator Library File. As such, a navigation aid can be provided e.g. to assist a clinician during treatment administration, or to enable computer assisted intervention in which following the optimal planned trajectory may be performed at least partially automatically.

Known markers are mainly designed for improved visibility in CT imaging and/or MRI. As another example, WO2020263092 describes a marker that has visibility in CT as well as MRI, with the advantage that a device equipped with such markers can be used in both types of imaging apparatuses. In other examples, the source path can be identified with markers forming a unique identification pattern, such that when the radioactive source is introduced into a target area via multiple channels, each channel can be identified.

Having visible markers, however, may cause artefacts in the CT and/or MR image, which adversely affects the definition of the 3-dimensional position of the visualized marker, and as such, its relation to the optimal planned trajectory. For the same reason, the number of markers that the radioactive source can practically be equipped with may be limited, to avoid having overexposed images. Accordingly, the resolution of determining the position and orientation of a radioactive source, e.g. to follow a curve on a multi-curved line, is currently limited as well.

As a result, the limited resolution of determining the source path that can be achieved during treatment administration may need to be accounted for when defining an optimal planned trajectory, e.g. by defining larger safety margins from healthy tissue structures or between trajectories, or by defining simpler yet more damaging trajectories, thereby making the radioactive source take a longer or suboptimal route to the target area.

The presence of artefacts, especially in case of multiple markers or source paths, may also reduce the ability to detect and distinguish between other features in the CT or MR image, such as tissue boundaries and geometrical features of the applicator or markers, such as edges and identifier patterns.

Accordingly, besides having limited ability to reconstruct a planned trajectory of a radioactive source, it can be a challenge to detect and manipulate a radioactive source with respect to other features, either in a manual or computer assisted fashion, due to loss of discriminatory capacity. As such, the level of uncertainty during treatment administration may be too high to allow manual reconstruction of a planned trajectory, in which the source path may be drawn manually by connecting the detected marker points, or automated reconstruction of a planned trajectory, in which the applicator model can be taken from an Applicator Library File and mapped onto the detected marker points, after which the source path of the mapped applicator model can be used as the optimal planned trajectory.

It is an object of the present invention to advance the field of marking arrangements for medical devices by addressing these and further disadvantages.

### SUMMARY

Aspects of the invention pertain to an arrangement for marking positions of a medical device along a source trajectory. The arrangement comprises a plurality of markers, arranged in series along the source trajectory. Each marker of the plurality of markers is aligned with the source trajectory, is made of a first material and comprises a coating. The coating is made of a second material disposed on a surface of the marker. A thickness of the coating is smaller than 1 micrometer, and an interdistance between each marker of the plurality of markers is smaller than 3 millimeter.

Accordingly, by having a sub-micron coating thickness, thereby minimizing the size of imaging artefacts, markers can be serially arranged along the source trajectory with a smaller interdistance between markers compared to conventional marking arrangements, to visualize the device path with improved resolution, such that a planned trajectory can be reconstructed with improved accuracy, e.g. to minimize safety margins and to enable treatment of poorly accessible target areas, and to increase visual discriminatory capacity to provide improved treatment performance, e.g. dose distribution of a radioactive source, during manual or automated reconstruction of a planned trajectory.

In some embodiments, the plurality of markers is arranged for forming an identifier pattern along the source trajectory. As such, individual source trajectories can be distinguished from a plurality of source trajectories e.g. in case multiple radioactive sources are to be placed in or near a target area.

In other or further embodiments, at least one marker of the plurality of markers is arranged for indicating a position of a source on the source trajectory. As a result, the marker arrangement can be used to accurately define the distance to dwell position, e.g. to adjust the radiation dose accordingly.

In yet other or further embodiments, the source trajectory spans a trajectory length, the plurality of markers span a marking length, and the marking length spans at least 50% of the trajectory length, preferably at least 80% of the trajectory length. In this way, markers are provided along a significant length of the source trajectory, such that a planned trajectory can be reconstructed with improved accuracy, e.g. to have enough visible points to define an entire lumen inside a patient.

In some embodiments, each marker comprises a ring coaxially aligned with the source trajectory, wherein the coating is disposed on a surface of the ring. Accordingly, the plurality of markers can e.g. be arranged along the inside of a brachytherapy applicator such that a clearance is provided for guiding a radioactive source through the applicator, or the plurality of markers can e.g. be arranged along the outside, e.g. around a catheter needle, or guide wire.

In some variants of these embodiments, the ring has an axial length ranging between 2 and 3 millimeter. By having these dimensions, a high-intensity and geometrically accurate signal can be provided in CT and MR imaging devices, that allows reducing corresponding imaging artefacts in size while the accuracy of the visible source path can be increased. For example, by having an axial length smaller than 3 millimeter, the signal produced by a marker allows the marker to be placed closer to an adjacent marker without creating artefacts that reduce the discriminatory properties of the individual markers. In this way, a higher resolution for (manual) reconstruction of the source path can be provided.

In other or further variants of these embodiments, the ring has an outer diameter smaller than 2 millimeter, in particular smaller than 1 millimeter. By having these dimensions, the marker can be used for smaller lumens and a further reduction of imaging artefacts in CT and MR imaging devices can be realized, while still providing a high-intensity and geometrically accurate signal.

In yet other or further variants of these embodiments, the ring has a wall thickness smaller than 0.3 millimeter, in particular smaller than 0.2 millimeter. As such, the marker arrangement can be coupled to a brachytherapy catheter or needle, while still providing a high-intensity and geometrically accurate signal and reducing the size of corresponding imaging artefacts in CT and MR. By thus limiting the extension of the marker arrangement in a direction lateral to that of the source trajectory, the diameter of the source trajectory can be decreased, which may provide for increased accuracy during manual or automated reconstruction.

In some embodiments, the inner ring is arranged for being visible in a T2 weighted MR imaging sequence comprising a magnetic field of at least 1.5 Tesla and a slice thickness of at least 2 millimeter, preferably at least 3 millimeter. Clinical guidelines, such as the GEC-ESTRO guidelines, recommend T2MRI for contouring of tumor and organs at risk, e.g. because of its higher soft-tissue resolution than T1MRI. As such, by being compatible with T2MRI imaging sequences, the marker arrangement can be visualized during diagnostic and/or clinical procedures, e.g. without requiring adjustment to conventional imaging equipment.

In some embodiments, the first material comprises one or more of copper, brass, bronze, phosphor, gold, silver, or titanium. Accordingly, the inner ring can be visualized in CT imaging devices.

In other or further embodiments, the second material comprises one or more of nickel or iron oxide, for being visible in an MR imaging sequence. Accordingly, the coating can be visualized with MR imaging devices.

Other aspects of the present invention relate to a medical device, comprising the marking arrangement according to claim 1. Beneficially, a source trajectory of the medical device can be visualized with improved resolution, by having a marking arrangement with smaller imaging artefacts and a smaller interdistance between markers, such that a planned trajectory can be reconstructed with improved accuracy, e.g. to minimize safety margins and to enable treatment of poorly accessible target areas, and to increase visual discriminatory capacity to provide improved performance during manual or automated reconstruction of a planned trajectory.

In some embodiments of the medical device, the marking arrangement is coupled to a brachytherapy applicator and/or a brachytherapy needle and/or a brachytherapy catheter. As such, the marking arrangement can be used to reconstruct a planned trajectory of a radioactive source during brachytherapy treatment with improved accuracy.

As will be described below, yet other aspects of the present invention relate to a system for reconstructing a planned trajectory of a medical device. The system comprises a detection module, arranged for detecting a marking arrangement as described herein, and a treatment calculation module, arranged for determining trajectories and dwell times, and comprising a controller and a non-transitory computer readable memory storing instructions thereon that, when executed by the controller, cause the controller to: determine a trajectory of the medical device based on the detected marking arrangement; compare, e.g. map, the determined trajectory of the medical device with the planned trajectory; and adjust a dwell time of the determined trajectory based on the comparison. Accordingly, by having a marking arrangement with smaller imaging artefacts and a smaller interdistance between markers, the trajectory of the medical device can be detected with improved resolution and accuracy, such that deviations of the device trajectory from the planned trajectory can be determined with improved accuracy to control the dwell time of the planned trajectory, thereby optimizing treatment administration e.g. by minimizing overexposure of healthy tissue or underexposure of the target area during brachytherapy treatment.

To distinguish between individual source trajectories in a plurality of source trajectories e.g. in case multiple radioactive sources are to be placed in or near a target area, the instructions, when executed by the controller, can further cause the controller to identify an identifier pattern based on the detected marking arrangement.

By having the instructions, when executed by the controller, further cause the controller to verify the identifier pattern against a stored identifier pattern associated with the planned trajectory, the determined trajectory can be coupled to a medical device associated with the stored identifier pattern, e.g. to rule out visualization and/or measurement errors.

In some embodiments of the system, the instructions, when executed by the controller, may cause the controller to compare the determined trajectory of the medical device with the planned trajectory, by measuring a deviation between detected marker positions on the determined trajectory and expected marker positions on the planned trajectory. As such, a detected position of a detected marker, e.g. comprising one or more measured values for translational and/or rotational degrees of freedom, can be compared to an expected position of corresponding markers on the planned trajectory, e.g. by a mapping comprising one or more corresponding expected values for translational and/or rotational degrees of freedom, to have a local comparison, e.g. local mapping, between the determined trajectory and the planned trajectory.

In other or further embodiments, the instructions, when executed by the controller, may further cause the controller to measure a margin between detected marker positions on the determined trajectory and surrounding tissue structures and/or additional detected marker positions. In this way, the marking arrangement can e.g. be used for measuring a safe distance between a radioactive source and surrounding healthy tissue structures or other radioactive sources, to avoid overtreatment or interference.

### BRIEF DESCRIPTION OF THE DRAWINGS

The invention will be further elucidated in the figures:

FIG. 1 illustrates a marking arrangement for marking positions of a medical device;

FIGS. 2A and 2B illustrate other or further embodiments of the marking arrangement;

FIGS. 3A-D provide detailed views of various embodiments of a marker 100 of the marking arrangement;

FIGS. 4A and 4B illustrate embodiments of a medical device, comprising the marking arrangement.

FIG. 5 represents an embodiment of a method for reconstructing a planned trajectory of a medical device;

FIG. 6 represents another or further embodiment of the method;

FIG. 7 represents yet another or further embodiment of the method;

FIG. 8 illustrates a system for reconstructing a planned trajectory of a medical device.

### DETAILED DESCRIPTION

The invention is described more fully hereinafter with reference to the accompanying drawings, in which embodiments of the invention are shown. In the drawings, the absolute and relative sizes of systems, components, layers, and regions may be exaggerated for clarity. Embodiments may be described with reference to schematic and/or cross-section illustrations of possibly idealized embodiments and intermediate structures of the invention. In the description and drawings, like numbers refer to like elements throughout. Relative terms as well as derivatives thereof should be construed to refer to the orientation as then described or as shown in the drawing under discussion. These relative terms are for convenience of description and do not require that the system be constructed or operated in a particular orientation unless stated otherwise.

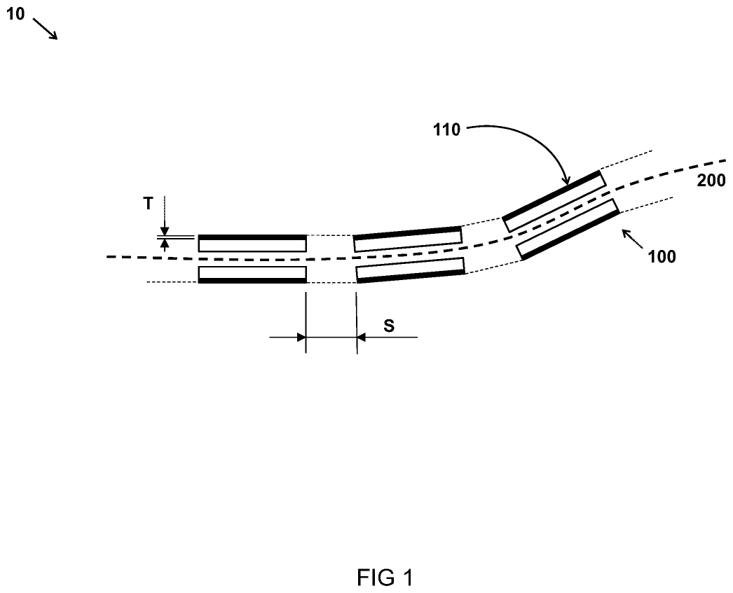

Turning to FIG. 1, there is illustrated a marking arrangement 10 for marking positions of a medical device, such as a brachytherapy applicator, catheter or needle, along a source trajectory 200. The source trajectory can for example be a straight or curved trajectory, for positioning a radioactive source in or near a target area, e.g. a tumour, inside the body of a patient. The marking arrangement 10 comprises a plurality of markers, arranged in series along the source trajectory 200 and each marker 100 of the plurality of markers is aligned with the source trajectory 200. For example, each marker 100 can be serially coupled along the length of an applicator or catheter, forming a series of markers, and each marker 100 can e.g. be aligned at least partially concentrically or at a distance or angular offset from the source trajectory 200. Each marker 100 is further made of a first material and comprises a coating 110 made of a second material disposed on a surface of the marker 100. For example, each marker 100 can be made of a CT visible material, such as a material comprising copper, brass, bronze, phosphor, gold, silver, titanium, or a combination thereof. The coating 110 can for example be made of an MR visible material, such as a material comprising nickel, iron, or iron oxide, or a combination thereof. To minimize the presence of imaging artefacts, the thickness T of the coating 110 can be reduced. In preferred embodiments, the thickness T of the coating 110 is smaller than 1 micrometer. For example, the thickness T can range between 0.1-1 micrometer, preferably between 0.1-0.5 micrometer. As shown in FIG. 1, each marker 100 is separated by an interdistance S. Synergistically, by having a coating thickness T smaller than 1 micrometer to minimize the presence of artefacts, the interdistance S between markers 100 can be reduced to enable visualizing the source trajectory with improved resolution. In preferred embodiments, the interdistance S between each marker 100 may be smaller than 3 millimeter. The interdistance S can for example range between 1-3 millimeter, preferably between 2-3 millimeter. In this way, compared to conventional markers which produce relatively larger imaging artefacts, the marking arrangement 10 described herein allows more markers 100 to be placed along the source trajectory 200. Accordingly, equipping a medical device with the marking arrangement 10 can improve the resolution of visualizing a source trajectory during treatment administration, such that a planned trajectory can be reconstructed with improved accuracy, which in turn may allow reducing safety margins, e.g. to surrounding tissue structures, during treatment planning. Alternatively, the marker arrangement 10 can be used as an individual accessory rather than being associated with a rigid or non-rigid medical device.

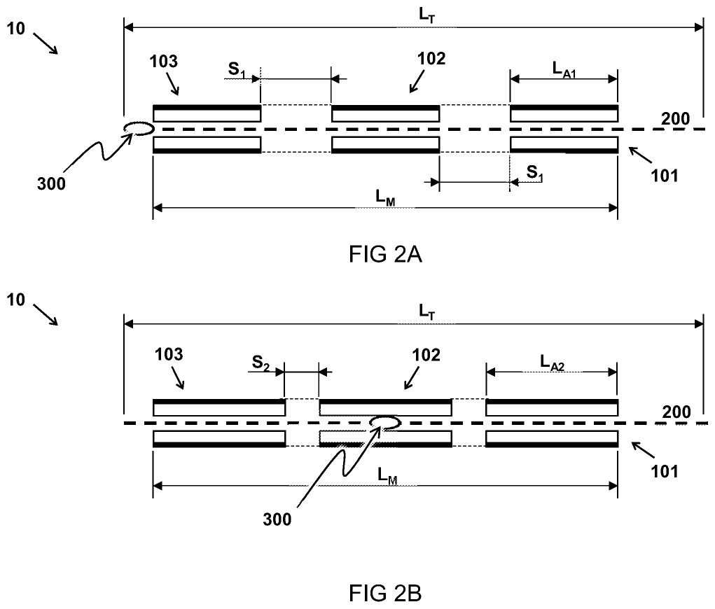

FIGS. 2A and 2B illustrate other or further embodiments of the marking arrangement 10 in which at least one of the markers 101, 102, 103 is arranged for indicating a position of a radioactive source 300 on the source trajectory 200. For example, as shown in FIG. 2A, marker 103, e.g. the most distal marker on the source trajectory 200 inside the body of a patient, can be arranged for indicating the position of the radioactive source 300, for example when the radioactive source 300 is intended to be positioned at the end of the source trajectory 200. As such, marker 103 can be used to indicate a predefined source position and to indicate the distance to dwell position zero, e.g. at the end of the source trajectory 200.

Alternatively, e.g. as shown in FIG. 2B, another marker can be arranged for indicating the position and/or distance to dwell position of a radioactive source 300, such as marker 102. In such cases, during treatment administration the radioactive source 300 may be positioned along the source trajectory 200 until a marker, such as marker 102 or marker 101, indicates that the radioactive source has reached its predefined source position and distance to a dwell position, e.g. to step-wise control the position of the radioactive source 300. As such, the improved marking resolution provided by the marking arrangement 10 described herein enables to more accurately measure and control the position of a radioactive source along a source trajectory.

As indicated in FIGS. 2A and 2B, the source trajectory 200 may span a trajectory length L_{T}, e.g. extending between the skin surface of a patient and a target area inside the body of the patient, while the plurality of markers may span a marking length L_{M}, e.g. extending between a most proximal marker, such as marker 101, and a most distal marker along the source trajectory 200, such as marker 103. Preferably, the marking length L_{M} spans at least 50% of the trajectory length L_{T}. For example, the marking length L_{M} can span between 50-100% of the trajectory length L_{T}, to enable visualization of at least 50% of the trajectory length L_{T}. More preferably the marking length L_{M} spans at least 80% of the trajectory length L_{T}, for example between 80-100% of the trajectory length L_{T}, to have a serial arrangement of markers providing visualization of at least 80% of the trajectory length L_{T}.

In some embodiments, the plurality of markers can be arranged for forming an identifier pattern along the source trajectory 200. An identifier pattern can for example be formed by having a specific combination of interdistances S between markers and axial lengths L_{A} of markers, e.g. to form a unique identifier pattern associated with a certain medical device, such as a brachytherapy applicator, needle or catheter. Alternatively, or additionally, the identifier pattern can be used to distinguish between multiple source paths within one or more medical devices. For example, as shown in FIG. 2A, a first medical device may be equipped with a marking arrangement 10 in which each marker is separated by a first interdistance S₁ and has a first axial length L_{A1}. As another example, e.g. as shown in FIG. 2B, a second medical device may be equipped with a marking arrangement 10 in which each marker is separated by a second interdistance S₂ and has a second axial length L_{A2}. The first interdistance S₁ can for example be different from the second interdistance S₂, and the first axial length L_{A1} can e.g. be different from axial length L_{A2}, such that the identifier patterns associated with the first medical device is different from the identifier pattern associated with the second medical device. In this way, a medical device, or the source trajectory, can be associated with a unique identifier pattern by the marking arrangement 10. Alternatively, or additionally, the identifier pattern can be formed by having a non-uniform interdistance, e.g. S1 or S2, along the source trajectory 200, and/or by having a non-uniform axial length, e.g. L_{A1} or L_{A2}, along the source trajectory 200.

To minimize the effect of additional imaging artefacts, e.g. areas of relatively high or low intensity, due to interference of artefacts produced by adjacent markers, the interdistance S between markers can be uniform along the source trajectory 200, e.g. having a uniform interdistance S within 10%, preferably within 5% along the source trajectory 200. For example as shown in FIG. 2A, the first interdistance S₁ between marker 101 and marker 102 can be approximately identical to the interdistance between marker 102 and marker 103, e.g. with a deviation ranging between 0-10%.

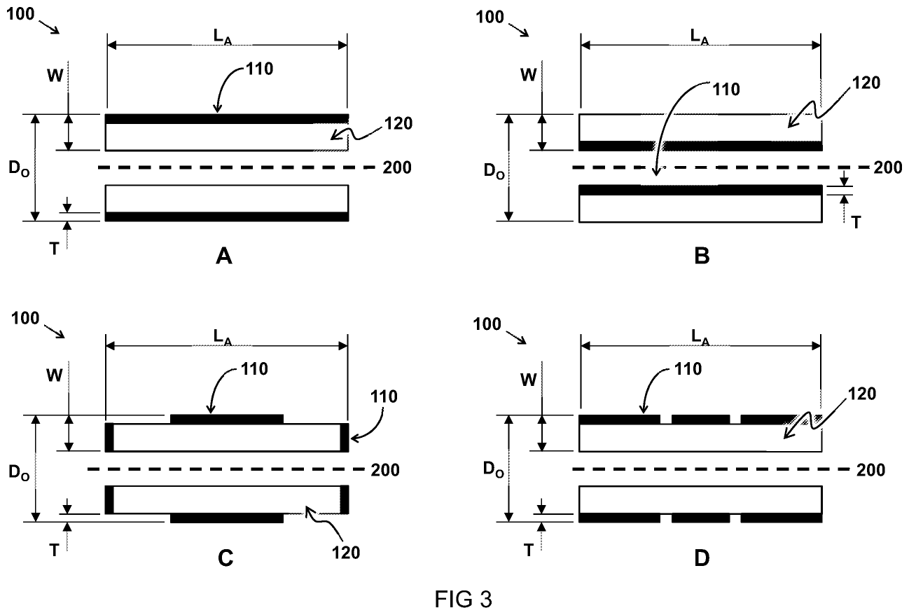

FIGS. 3A-D provide detailed views of various embodiments of a marker 100 of the marking arrangement describe herein, in which the marker 100 comprises a ring 120 coaxially aligned with the source trajectory 200, and in which the coating 110 is disposed on a surface of the ring 120. The coating 110 can for example be disposed on an outer cylindrical surface of the ring 120, as shown in FIGS. 3A and 3D, with the coating 110 covering at least part of the outer cylindrical surface. Alternatively, e.g. as shown in FIG. 3B, the coating 110 can at least partially cover an inner cylindrical surface of the ring 120. Alternatively, e.g. as shown in FIG. 3C, the coating 110 can at least partially cover one or more axial end surfaces of the ring 120. Alternatively, or additionally, in some variants of the marker 100 the ring 120 may comprise inner and outer cylindrical surfaced with a concave or convex radius of curvature, e.g. to increase visibility of the ring 120 and/or the coating 110 in imaging devices, and to smoothen the edges of the marker e.g. to minimize obstruction and/or damage during use. Combinations of the embodiments depicted in FIGS. 3A-D and described above can be envisioned by the person skilled in the art,

As depicted in FIG. 3D, the coating 110 can be disposed on a surface of the ring 120, e.g. an inner surface or an outer surface, such that a pattern is formed by the coating, e.g. to have a unique identifier pattern provided by an individual or stand-alone marker 100. The pattern can for example be an alternating pattern of coated and non-coated parts of the ring 120. As such the pattern can e.g. be used as part of an identifier pattern for the marking arrangement. The pattern can for example be formed in one dimension, e.g. along or transverse to the source trajectory 200, in two dimensions, e.g. forming a checkerboard pattern, or in three dimensions, e.g. by having a varying coating thickness T along the coated surface.

As shown in FIGS. 3A-D, the marker 100 has an axial length L_{A} spanning between axial ends of the ring 120. The marker 100 may also have an outer diameter D_{O} diametrically spanning an outer cylindrical surface of the ring 120. Because of the relatively small thickness of the coating 110 relative to the wall thickness W and the outer diameter D_{O} of the ring, the wall thickness W and the outer diameter D_{O} may include the coating thickness T, e.g. as shown in FIGS. 3A-D.

Preferably, the ring 120, e.g. the coating 110, is arranged for being visible in a T2 weighted MR imaging sequence comprising a magnetic field of at least 1.5 Tesla and a slice thickness of at least 2 millimeter, preferably a slice thickness of at least 3 millimeter, to be able to visualize the marking arrangement during diagnostic and/or clinical procedures, using conventional imaging equipment and standardized visualization protocols. Additionally, the ring is arranged for being visible in CT imaging devices. Dimensions of the ring 120 and its coating 110, such as the axial length L_{A}, the outer diameter D_{O}, the wall thickness W and the coating thickness T, can for example be designed accordingly, to have the marker produce a signal that can be optimally visualized in the above described MR imaging sequence and in CT imaging devices. In some embodiments, the first material of the marker 100, e.g. the material of the ring 120, comprises one or more of copper, brass, bronze, phosphor, gold, silver, or titanium. These materials are known to provide visibility in CT imaging devices. In other or further embodiments, the second material of the marker 100, i.e. the material of the coating 110 comprises one or more of nickel or iron oxide, for being visible in an MR imaging sequence.

Preferably, the ring 120 has an axial length L_{A} ranging between 2 and 3 millimeter. For example, the axial length L_{A} is between 2.2 and 2.8 millimeter, or approximately 2.5 millimeter. Preferably, the axial length L_{A} is larger than 2 millimeter, to produce a signal with an intensity high enough for visualization by CT and/or MR imaging because it is related to the nickel area. Conversely, the axial length L_{A} is preferably smaller than 3 millimeter, to have a geometrically accurate signal that allows the marker to be placed more closely to another marker, thereby providing more maker points and producing a higher resolution for (manual) reconstruction of the source path.

In other or further preferred embodiments, the ring 120 has an outer diameter D_{O} smaller than 2 millimeter. For example, the outer diameter D_{O} can be between 0.3 and 2 millimeter. In particular, the outer diameter is preferably smaller than 1 millimeter. In this way, the according size of the marker synergistically allows reducing the inner diameter of the medical device, such as an applicator, thereby allowing to reduce the size of the lumen inside the body of a patient during treatment administration and produces smaller imaging artefacts compared to conventional markers, while still generating a high intensity and geometrically accurate signal in CT and MR imaging devices.

In yet other or further preferred embodiments, e.g. as shown in FIGS. 3A-D, the ring 120 has a wall thickness W smaller than 0.3 millimeter. For example, the wall thickness can range between 0.05-0.3 millimeter, or between 0.1-0.2 millimeter. In particular embodiments, the wall thickness W may be smaller than 0.2 millimeter. Accordingly, in combination with the thickness T of the coating 110 being smaller than 1 micrometer, thus at least a factor 50 smaller than the wall thickness W of the ring, the marker 100 can be optimally dimensioned to produce a high intensity and geometrically accurate signal in CT and/or MR imaging devices while minimizing the presence of imaging artefacts. Synergistically, having a wall thickness W smaller than 0.3 millimeter may limit the extension of the marker 100 in a direction lateral to the source trajectory 200, which e.g. allows that the marker 100 can be coupled to the external surface of a medical device, such as a brachytherapy catheter or needle, without significantly increasing the size of the medical device and/or without forming an obstruction. Moreover, by having a wall thickness W smaller than 0.3 millimeter, the diameter of the source channel can be decreased which in turn my increase the accuracy of the visualization and/or the reconstruction of the source trajectory.

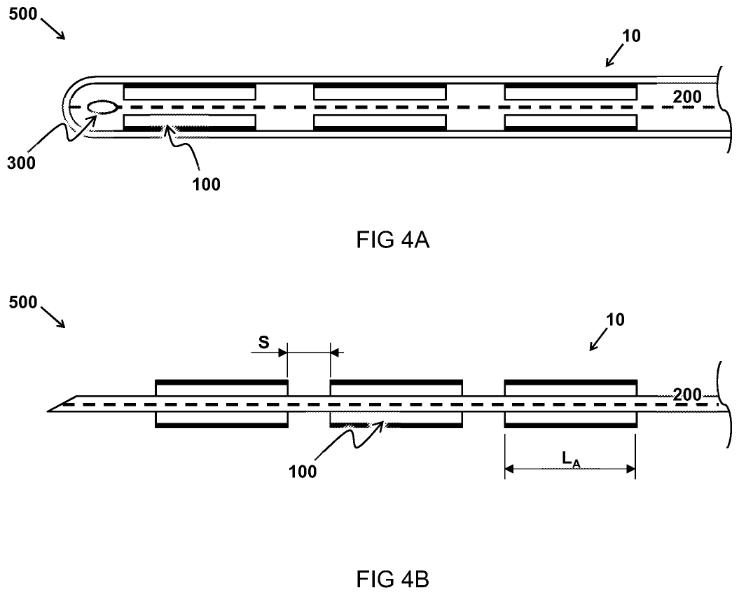

FIGS. 4A and 4B illustrate embodiments of a medical device 500, comprising the marking arrangement 10 described herein. The marking arrangement 10 can be coupled to a relatively rigid medical device 500, such as a brachytherapy applicator, e.g. on the inside of the applicator tube as shown in FIG. 4A. The markers 100 of the marking arrangement can for example be mounted on an inner surface of the brachytherapy applicator along the source trajectory 200. When the markers 100 are ring shaped, e.g. as shown in FIG. 4A, a radioactive source 300 may be able to pass along the source trajectory through the inner hole of the markers 100. Alternatively, the markers 100 can e.g. have a partially circular shape, having an outer contour at least partially following the inner contour of the brachytherapy applicator and an inner contour providing clearance for passing a radioactive source.

In other or further embodiments, e.g. as shown in FIG. 4B, the marking arrangement 10 can be coupled to a relatively flexible, non-rigid, medical device 500, such as a brachytherapy needle and/or a brachytherapy catheter. In these configurations, the markers 100 can e.g. be mounted along the source trajectory 200 on an external surface of the medical device 500. When the markers are ring shaped, or at least partially ring shaped, the inner hole of the markers can be used for mounting to the medical device 500, as shown in FIG. 4B. Alternatively, the marker cross section can have any other geometry, e.g. rectangular, triangular, hexagonal, etc. Along the axial direction, the markers may not necessary have a straight and uninterrupted contour, but can e.g. have a surface that is grooved, recessed, or provided with holes. Equipping the medical device 500 with the marking arrangement 10 may cause a reduced flexibility of the medical device 500. By optimizing the axial length L_{A} of the markers 100 and the interdistance S between markers 100, the reduction in flexibility can be limited, while improving the visualization resolution of the source trajectory e.g. by having an increased number of markers 100 with a smaller axial length L_{A} and at a smaller interdistance S. As such, a medical device 500 equipped with the marking arrangement 10 can be used to reconstruct a planned trajectory of a radioactive source during brachytherapy treatment with improved accuracy during treatment administration, which in turn may allow minimizing safety margins during treatment planning.

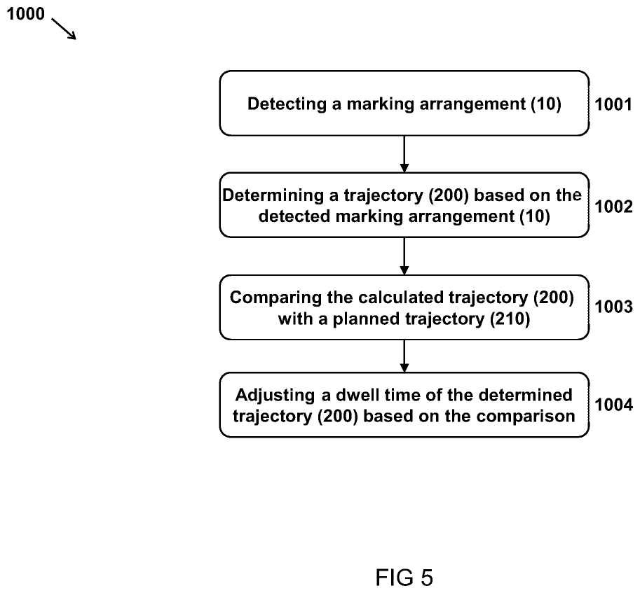

FIGS. 5-7 represent embodiments of a method 1000 for manual or automated reconstruction of a planned trajectory 210 of a medical device, such as a brachytherapy applicator, catheter or needle, equipped with a marking arrangement as described herein. During manual reconstruction, the source path may be identified manually by following the detected marker points. During automated reconstruction, an applicator model may e.g. be placed from an Applicator Library File onto the detected marker points, and the source path of the mapped applicator model can then be used as the optimal planned trajectory. In some embodiments, e.g. as shown in FIG. 5, the method 1000 comprises detecting the marking arrangement (step 1001), determining a trajectory 200 of the medical device based on the detected marking arrangement 10 (step 1002), comparing the determined trajectory 200 of the medical device with the planned trajectory 210 (step 1003), and adjusting a dwell time of the determined trajectory 200 based on the comparison (step 1004). Use of the marking arrangement 10 as described herein allows detecting the trajectory 200 of the medical device with improved resolution and accuracy, by the marker arrangement 10 providing smaller detection points and therefore more points to determine and (manually) reconstruct the source trajectory. During automated reconstruction, an applicator model, e.g. taken from an Applicator Library File, can be mapped with improved precision on the detected marker points, thereby providing a better comparison, e.g. mapping, between the determined trajectory and the planned trajectory. As a result, improved identification of the dwell position of the planned trajectory can be provided both for manual and for automated reconstruction, e.g. to optimize treatment administration.

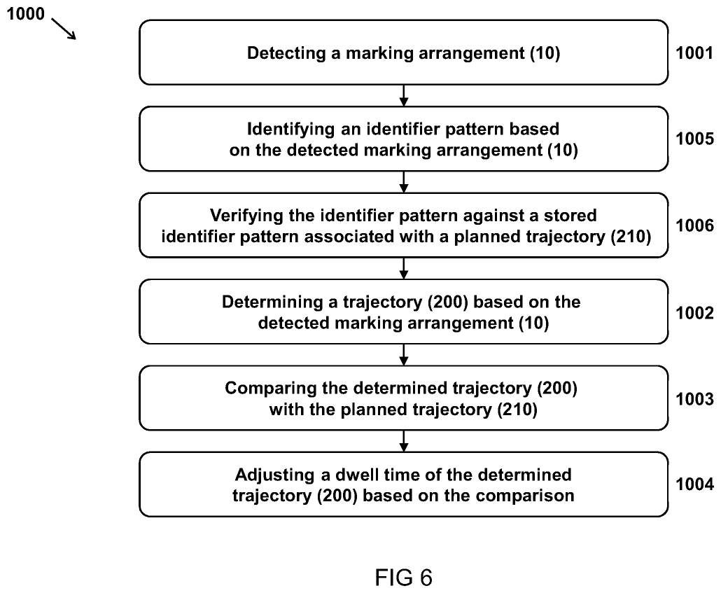

In other or further embodiments, e.g. as shown in FIG. 6, the method 1000 may further comprise step 1005 of identifying an identifier pattern based on the detected marking arrangement 10, and step 1006 of verifying the identifier pattern against a stored identifier pattern associated with the planned trajectory 210, prior to step 1002 of determining the trajectory 200 based on the detected marking arrangement 10. Accordingly, the marking arrangement 10 can be used to distinguish between individual source trajectories, e.g. each coupled to a medical device associated with a stored unique identifier pattern, in a plurality of source trajectories e.g. in case multiple source paths are to be placed in or near a target area.

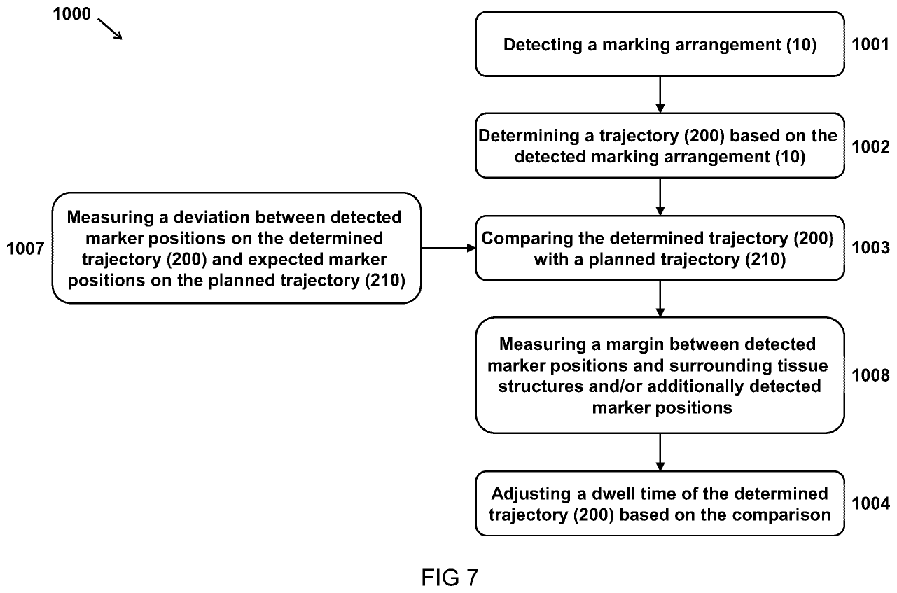

In some embodiments of the method 1000, e.g. as shown in FIG. 7, step 1003 of comparing comprises step 1007 of measuring a deviation between detected marker positions on the determined trajectory 200 and expected marker positions on the planned trajectory 210. A detected marker position can e.g. comprise a measured position in an x-direction, a y-direction and/or a z-direction, for defining a three dimensional position of the detected marker, e.g. relative to a reference frame or a reference marker. Additionally, or alternatively, the detected marker position can e.g. comprise a measured angle around a pitch axis, a yaw axis, and/or a roll axis, for defining a three dimensional orientation of the detected marker, e.g. relative to the reference frame or a reference marker. The measured position and/or orientation of the detected marker can be compared to an expected position and/or orientation of a corresponding expected marker on the planned trajectory. Accordingly, one or more local comparisons, e.g. local mappings, can be made between detected and expected markers along the determined and planned trajectory, respectively, to improve the comparison. In other or further embodiments, the method 1000 further comprises step 1008 of measuring a margin between detected marker positions on the determined trajectory 200 and surrounding tissue structures and/or additionally detected marker positions. Accordingly, the measured margin can e.g. be used for defining a safe distance between a radioactive source positioned on or along the determined trajectory 200 and surrounding healthy tissue structures, e.g. produced by the MR images, or a safe distance between the determined trajectory 200 and planned trajectories of other radioactive sources, e.g. positioned in or near the target area by other medical devices, to avoid overtreatment.

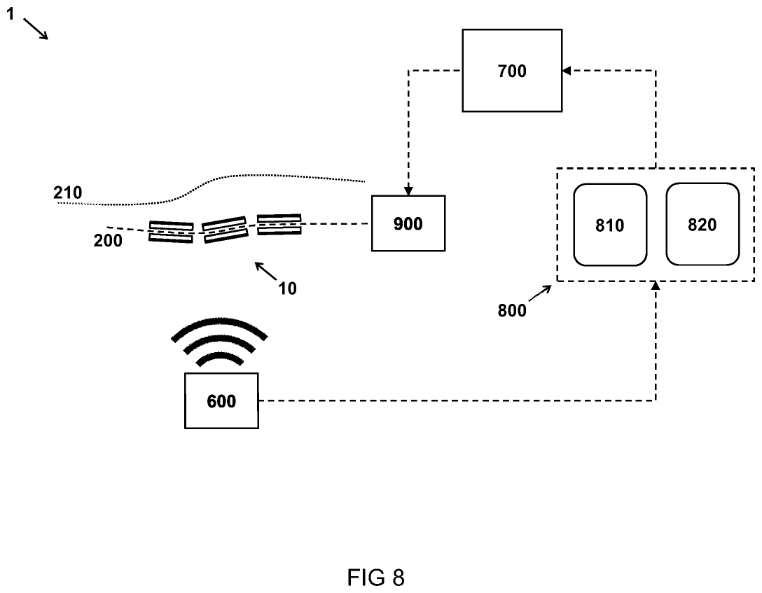

FIG. 8 illustrates a system 1 for reconstructing a planned trajectory 210 of a medical device, such as a brachytherapy applicator, catheter or needle. The system 1 comprises a detection module 600, e.g. comprising an MR and/or CT imaging device, and arranged for detecting a marking arrangement 10 as described herein. The system further comprises a treatment calculation module 800, arranged for determining trajectories, dwell positions and dwell times. The treatment calculation module 800 comprises a controller 810. The controller 810 may comprise one or more computers or processors and can be in wired, wireless, direct, or networked communication with other components and modules of the system 1, such as the detection module 600. The controller 810 can also be remotely hosted in a cloud server. The treatment calculation module further comprises a non-transitory computer readable memory 820, e.g. a hard disk drive, a DVD, a CD, flash memory, read-only memory, random-access memory, etc. The non-transitory computer readable memory 820 stores instructions thereon, such as program code in the form of e.g. software and/or firmware. The instructions may cause the controller 810 to determine the trajectory 200 of the medical device based on the detected marking arrangement 10, which allows reconstructing the trajectory with improved accuracy. The instructions can further cause the controller 810 to compare the determined trajectory 200 with the planned trajectory 210, e.g. to control process parameters. As a result of the improved accuracy during the detection of the marking arrangement, the comparison, e.g. mapping, between the determined trajectory 200 and the planned trajectory 210 can be improved as well. As such, treatment administration can e.g. be optimized by minimizing overexposure of healthy tissue or underexposure of the target area during brachytherapy treatment, by having the instructions cause the controller 810 to adjust a dwell time according to the improved mapping.

In some embodiments of the system 1, the instructions, when executed by the controller 810, may further cause the controller 810 to identify an identifier pattern based on the detected marking arrangement 10. The identifier pattern can for example be used to distinguish between individual source trajectories, e.g. each coupled to a medical device associated with a stored unique identifier pattern associated with the planned trajectory 210. The instructions may further cause the controller 810 to verify the identifier pattern against the stored identifier pattern, e.g. to identify artefact caused by visualization and/or measurement errors that are not part of any stored identifier pattern, and e.g. to distinguish a particular trajectory in case multiple radioactive sources are to be placed in or near a target area.

In other or further embodiments, the instructions may cause the controller 810 to compare the determined trajectory 200 with the planned trajectory 210 by measuring a deviation between detected marker positions on the determined trajectory 200 and expected marker positions on the planned trajectory 210. For example, a detected marker position can comprise one or more measured degrees of freedom, including positional degrees of freedom along an x-axis, y-axis, and/or a z-axis, and/or including angular degrees of freedom around a roll axis, pitch axis, and/or a yaw axis. A detected marker position can for example be measured relative to a reference frame, relative to another marker, or relative to the planned trajectory 210. The instructions may further cause the controller 810 to compare the measured position and/or orientation of the detected marker to an expected position and/or orientation of a corresponding expected marker on the planned trajectory 210. In this way, the comparison between the determined and planned trajectory 200, 210 can be improved by having one or more local comparisons between detected and expected markers along the determined and planned trajectory 200, 210, respectively.

Additionally, or alternatively, e.g. to avoid overtreatment, the marking arrangement 10 can be used for measuring a safe distance, for example between a radioactive source and surrounding healthy tissue structures or other radioactive sources, by having the instructions cause the controller 810 to measure a margin between detected marker positions on the determined trajectory 200 and surrounding tissue structures and/or additional detected marker positions.

In some embodiments of the system 1, e.g. as shown in FIG. 8, the system 1 further comprises a communication module 700 arranged for communicating the determined trajectory 200 and dwell time to an afterloader system 900. The afterloader system 900 can e.g. be coupled to a brachytherapy applicator, catheter, or needle, equipped with a marking arrangement as described herein. Accordingly, process parameters of the afterloader system 900 can be adjusted, for example based on real-time data from the detection module 600 and the treatment calculation module 800, e.g. manually or (semi-) automatically. This may reduce the workload for an operator to accurately reconstruct a source trajectory and shorten the response time to adjust the afterloader system 900 to any deviations between the determined trajectory 200 and the planned trajectory 210.

It is thus believed that the operation and construction of the present invention will be apparent from the foregoing description and drawings appended thereto. For the purpose of clarity and a concise description features are described herein as part of the same or separate embodiments, however, it will be appreciated that the scope of the invention may include embodiments having combinations of all or some of the features described.

The invention applies not only to brachytherapy applications where the marking arrangement is used for marking a device trajectory, but also to other medical or industrial applications where a marking arrangement is used. It will be clear to the skilled person that the invention is not limited to any embodiment herein described and that modifications are possible which may be considered within the scope of the appended claims. Also kinematic inversions are considered inherently disclosed and can be within the scope of the invention. In the claims, any reference signs shall not be construed as limiting the claim.

The terms ‘comprising’ and ‘including’ when used in this description or the appended claims should not be construed in an exclusive or exhaustive sense but rather in an inclusive sense. Thus expression as ‘including’ or ‘comprising’ as used herein does not exclude the presence of other elements, additional structure or additional acts or steps in addition to those listed. Furthermore, the words ‘a’ and ‘an’ shall not be construed as limited to ‘only one’, but instead are used to mean ‘at least one’, and do not exclude a plurality. Features that are not specifically or explicitly described or claimed may additionally be included in the structure of the invention without departing from its scope.

Expressions such as: “means for . . . ” should be read as: “component configured for . . . ” or “member constructed to . . . ” and should be construed to include equivalents for the structures disclosed. The use of expressions like: “critical”, “preferred”, “especially preferred” etc. is not intended to limit the invention. To the extent that structure, material, or acts are considered to be essential they are inexpressively indicated as such. Additions, deletions, and modifications within the purview of the skilled person may generally be made without departing from the scope of the invention, as determined by the claims.

1. A marking arrangement for marking positions of a medical device along a source trajectory, the marking arrangement comprising:
a plurality of markers, arranged in series along the source trajectory, wherein each marker of the plurality of markers are aligned with the source trajectory, are made of a first material, and comprise a coating, wherein the coating is made of a second material disposed on a surface of each marker of the plurality of markers, wherein a thickness of the coating is smaller than 1 micrometer, and wherein an interdistance between each marker of the plurality of markers is smaller than 3 millimeters.

2. The marking arrangement according to claim 1, wherein the plurality of markers is arranged for forming an identifier pattern along the source trajectory.

3. The marking arrangement according to claim 1, wherein at least one marker of the plurality of markers is arranged for indicating a position of a radioactive source on the source trajectory.

4. The marking arrangement according to claim 1, wherein the source trajectory spans a trajectory length, wherein the plurality of markers span a marking length, and wherein the marking length spans at least 50% of the trajectory length.

5. The marking arrangement according to claim 1, wherein each marker comprises a ring coaxially aligned with the source trajectory, and wherein the coating is disposed on a surface of the ring.

6. The marking arrangement according to claim 5, wherein the ring has an axial length ranging between 2 and 3 millimeters.

7. The marking arrangement according to claim 6, wherein the ring has an outer diameter smaller than 2 millimeters.

8. The marking arrangement according to claim 5, wherein the ring has a wall thickness smaller than 0.3 millimeters.

9. (canceled)

10. The marking arrangement according to claim 1, wherein the first material comprises one or more of copper, brass, bronze, phosphor, gold, silver, or titanium.

11. The marking arrangement according to claim 1, wherein the second material comprises one or more of nickel or iron oxide, and is visible in a magnetic resonance (MR) imaging sequence.

12. A medical device, comprising a marking arrangement, wherein the marking arrangement comprises:
a plurality of markers arranged in series along a source trajectory, wherein each marker of the plurality of markers are aligned with the source trajectory, are made of a first material, and comprise a coating, wherein the coating is made of a second material disposed on a surface of each marker of the plurality of markers, wherein a thickness of the coating is smaller than 1 micrometer, and wherein an interdistance between each maker of the plurality of markers is smaller than 3 millimeters.

13. The medical device according to claim 12, wherein the marking arrangement is coupled to at least one of a brachytherapy applicator, a brachytherapy needle, or a brachytherapy catheter.

14. A system for reconstructing a planned trajectory of a medical device, the system comprising:
a marking arrangement comprising:
a plurality of markers arranged in series along a source trajectory, wherein each marker of the plurality of markers are aligned with the source trajectory. are made of a first material, and comprise a coating, wherein the coating is made of a second material disposed on a surface of each marker of the plurality of markers, wherein a thickness of the coating is smaller than 1 micrometer, and wherein an interdistance between each maker of the plurality of markers is smaller than 3 millimeters;
a detection module, arranged for detecting the marking arrangement; and
a treatment calculation module, arranged for determining trajectories and dwell times, wherein the treatment calculation module comprises a controller and a non-transitory computer readable memory storing instructions thereon that, when executed by the controller, cause the controller to:
determine a trajectory of the medical device based on the detected marking arrangement;
compare the determined trajectory of the medical device with the planned trajectory; and
adjust a dwell time of the determined trajectory based on comparing the determined trajectory of the medical device with the planned trajectory.

15. The system according to claim 14, wherein the instructions, when executed by the controller, further cause the controller to:
identify an identifier pattern based on the detected marking arrangement.

16. The system according to claim 15, wherein the instructions, when executed by the controller, further cause the controller to;
verify the identifier pattern against a stored identifier pattern associated with the planned trajectory.

17. The system according to claim 14, wherein the instructions, when executed by the controller, cause the controller to compare the determined trajectory of the medical device with the planned trajectory, by measuring a deviation between detected marker positions on the determined trajectory and expected marker positions on the planned trajectory.

18. The system according to claim 14, wherein the instructions, when executed by the controller, further cause the controller to measure a margin between detected marker positions on the determined trajectory and surrounding tissue structures and/or additional detected marker positions.

19. The system according to claim 14, further comprising:
a communication module arranged for communicating the determined trajectory and the dwell time to an afterloader system.

20. The marking arrangement according to claim 4, wherein the marking length spans at least 80% of the trajectory length.

21. The medical device according to claim 12, wherein the thickness of the coating and interdistance between markers are configured to minimize imaging artifacts while enabling visualization of the source trajectory in both computed tomography (CT) imaging and magnetic resonance (MR) imaging, wherein each marker of the plurality of markers comprises a ring coaxially aligned with the source trajectory, and wherein the ring is arranged for being visible in a T2 weighted MR imaging sequence comprising a magnetic field of at least 1.5 Tesla and a slice thickness of at least 2 millimeters.
